(19) United States
(12) Patent Application Publication
(10) Pub. No.: US 2025/0248841 A1
(43) Pub. Date: Aug. 7, 2025

(54) CONTAINER WITH ATTACHMENT MECHANISM FOR FACILITATING COLD THERAPY

(71) Applicant: ARCTIC SHOWER LLC, Syosset, NY (US)

(72) Inventor: John Bartholomew Quirk, III, Oyster Bay, NY (US)

(73) Assignee: ARCTIC SHOWER LLC, Syosset, NY (US)

(21) Appl. No.: 19/185,825

(22) Filed: Apr. 22, 2025

Related U.S. Application Data
(63) Continuation of application No. PCT/US24/52865, filed on Oct. 24, 2024.
(60) Provisional application No. 63/593,316, filed on Oct. 26, 2023.

(51) Int. Cl. A61F 7/10; A61F 7/00

(52) U.S. Cl. CPC A61F 7/103; A61F 2007/0063

(57) ABSTRACT

A container including an opening in a top portion sized and adapted to receive water from a water source; one or more side walls and a bottom wall below the opening, the one or more side walls and the bottom wall forming at least one interior compartment; a plurality of apertures on the bottom wall; and one or more fasteners in the top portion above the opening, the one or more fasteners being adapted to attach the container to the water source, wherein the one or more side walls incorporate an insulating material and the at least one interior compartment is thereby adapted to contain a cooling substance for cooling the water received from the water source and to allow cooled water to exit the at least one interior compartment through the plurality of apertures on the bottom wall.

### 1. CROSS-REFERENCE TO RELATED APPLICATIONS

The present application is a continuation of International Patent Application No. PCT/US2024/052865, filed Oct. 24, 2024, which claims benefit of and priority from U.S. Patent Application No. 63/593,316, filed on Oct. 26, 2023, the entirety of each of which is hereby incorporated by reference as if set forth herein.

### 2. FIELD

The present disclosure generally relates to a consumer product and, more specifically, to a container for a cooling substance that is adapted to attach to a water source so that water from the water source is cooled within the container, the container having a plurality of apertures to allow the cooled water to exit the container and thereby facilitate cold therapy.

### 3. BACKGROUND

Ice baths, also known as cold therapy or cryotherapy, have long been used to treat ailments and to promote physical well-being. The continuing developments and use of ice baths can be attributed to advancements in sports medicine and the increasing interest in recovery techniques among athletes. Some of the benefits of cold therapy include reducing inflammation, aiding in muscle recovery, and relieving pain. Consequently, ice baths are widely used for athletes' recovery routines, be it in professional sports, fitness centers, and rehabilitation clinics.

Typically, ice baths involve immersing the body in a tub filled with ice-cold water, maintained at temperatures between 50 to 59 degrees Fahrenheit (10 to 15 degrees Celsius). Durations for ice baths vary and can last between 10 to 20 minutes. For certain uses, such as casual exercise recovery, ice baths can involve higher temperatures and/or shorter durations.

Some of the known benefits of ice baths and cold therapy include reduction of inflammation and swelling, aid in flushing of waste products in fatigued muscles, stimulation of the immune system, and mental state improvement.

It is understood that the cold temperatures of an ice bath constrict blood vessels and reduce blood flow to the immersed areas of the body. This vasoconstriction helps in reducing inflammation and swelling, which is beneficial for post-exercise recovery or injury rehabilitation. Additionally, cold temperatures of an ice bath can numb the nerves, which may provide temporary pain relief and soothing of sore muscles. Ice baths can also aid in flushing out waste products, such as lactic acid, from the muscles and, thereby, accelerate the recovery of fatigued muscles. Thus, ice baths are commonly made available in professional athletic organizations to allow athletes to recover after intense training or competitions, and to improve subsequent performance.

Aside from its use in sports medicine, cold therapy has also been understood to enhance the immune system. As an example, it is believed that exposure to cold temperatures stimulates the production of white blood cells. Thus, cold therapy also been used to boost overall health and prevent illnesses.

Finally, ice baths have also gained attention for their potential mental health benefits. Similar to the immune response mentioned above, it is believed that an intense cold shock triggers a stress response in the body, which can aid in resilience and mental fortitude. Additionally, it is believed that rapid cooling of the body during an ice bath stimulates the release of endorphins, hormones associated with feelings of happiness and relaxation, thereby enhancing mood and reducing stress. Thus, ice baths have also been used as part of meditative practices for promoting mental clarity.

To create the conditions of an ice bath in a conventional bathtub at home, a dedicated chest freezer would be needed to produce an adequate amount of ice to fill the bathtub. Such a freezer would be in the hundreds to thousands of dollars and would require a large amount of space for this dedicated purpose. Furthermore, the prone position of a conventional bathtub does not conform to professional ice baths, which usually provide for a sitting position.

In view of the above, special purpose, at home, ice bathtubs have been marketed. Some such ice tubs include onboard cooling systems for providing a built-in source for ice cold water. Others have been more rudimentary sitting tubs that still require the user to provide the ice and the water to fill the sitting tubs.

### 4. SUMMARY

Given the multitude of recognized benefits of cold therapy, it is an object of the present disclosure to provide access to such benefits without the inordinate investment in equipment, and dedicated space, needed for an ice bath.

Thus, the present disclosure provides a container for a cooling substance that is adapted to attach to a water source so that water from the water source is cooled within the container, the container having a plurality of apertures to allow the cooled water to exit the container and thereby facilitate cold therapy.

In one example implementation of the present disclosure, an ice container with apertures is provided for attachment to a shower head. The container can be filled with ice, for example, from a conventional home freezer. As water from the shower fills the container, it is cooled by the ice in the container and the cooled water exits the container through the apertures to facilitate a cold therapy shower.

According to an example implementation of the present disclosure, a container, comprises an opening in a top portion sized and adapted to receive water from a water source; one or more side walls and a bottom wall below the opening, said one or more side walls and the bottom wall forming at least one interior compartment; a plurality of apertures on the bottom wall; and one or more fasteners in the top portion above the opening, the one or more fasteners being adapted to attach the container to the water source, wherein the one or more side walls incorporate an insulating material and the at least one interior compartment is thereby adapted to contain a cooling substance for cooling the water received from the water source and to allow cooled water to exit the at least one interior compartment through the plurality of apertures on the bottom wall.

According to one embodiment, the container further comprises a plurality of handles, wherein the one or more fasteners comprise a plurality of hook and loop fastener areas incorporated on respective ones of the plurality of handles of the container.

According to one embodiment, a first hook and loop fastener area is disposed on an inner side of a first one of the plurality handles and a second hook and loop fastener area is disposed on an outer side of a second one of the plurality of handles.

According to one embodiment, the plurality apertures are arranged in a radially symmetrical pattern.

According to one embodiment, the plurality of apertures are arranged in a radially aligned pattern.

According to one embodiment, the bottom wall is circular in shape.

According to one embodiment, the container further comprises one or more additional fasteners adapted to fasten to a structure proximate to the water source.

According to one embodiment, the one or more additional fasteners comprise one or more suction cups adapted to fasten to a wall proximate to the water source.

According to an example implementation of the present disclosure, a method of initiating a cold therapy shower using a container that comprises an opening in a top portion sized and adapted to receive water from a water source; one or more side walls and a bottom wall below the opening, said one or more side walls and the bottom wall forming at least one interior compartment; a plurality of apertures on the bottom wall; and one or more fasteners in the top portion above the opening, the one or more fasteners being adapted to attach the container to the water source, wherein the one or more side walls incorporate an insulating material and the at least one interior compartment is thereby adapted to contain a cooling substance for cooling the water received from the water source and to allow cooled water to exit the at least one interior compartment through the plurality of apertures on the bottom wall, said method comprises: placing a cooling substance into the at least one interior compartment through the opening; attaching the container to the water source using the one or more fasteners while aligning the opening to water source; and initiating a water flow from the water source into the opening.

According to one embodiment, the cooling substance comprises about 5 to 12 pounds of ice.

According to one embodiment, an entry temperature of the water flow from the water source is approximately 70 degrees Fahrenheit.

According to one embodiment, an exit temperature of cooled water flowing from at least one of the plurality of apertures of the container is approximately 50 degrees Fahrenheit.

According to one embodiment, the exit temperature is maintained for about 3 to 5 minutes.

According to one embodiment, the exit temperature is maintained for about 4 minutes.

### 5. BRIEF DESCRIPTION OF THE DRAWINGS

Various example implementations of this disclosure will be described in detail, with reference to the following figures, wherein:

FIG. 1A is a front side view illustrating a cooling substance container bag according to one example implementation of the present disclosure.

FIG. 1B is a rear side view showing the cooling substance container bag of FIG. 1A.

FIG. 1C is a top plan view showing the cooling substance container bag of FIG. 1A.

FIG. 1D is a bottom plan view showing the cooling substance container bag of FIG. 1A.

FIG. 2A is a procedural diagram showing the first two steps of a process for using the cooling substance container bag of FIG. 1A.

FIG. 2B is a procedural diagram showing a third step of the process of FIG. 2A.

FIG. 3 is a perspective view illustrating a cooling substance container bag according to another example implementation of the present disclosure.

FIG. 4A is a perspective view illustrating a cooling substance container bag according to yet another example implementation of the present disclosure.

FIG. 4B is a diagram of the cooling substance container bag of FIG. 4A in use in correspondence with the procedural diagram of FIG. 2B.

### 6. DETAILED DESCRIPTION

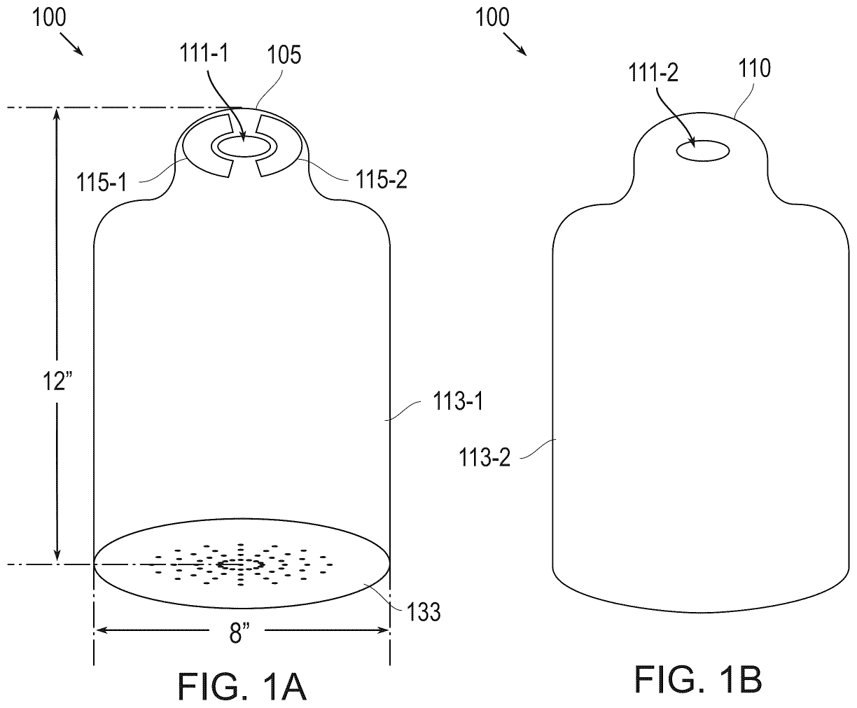

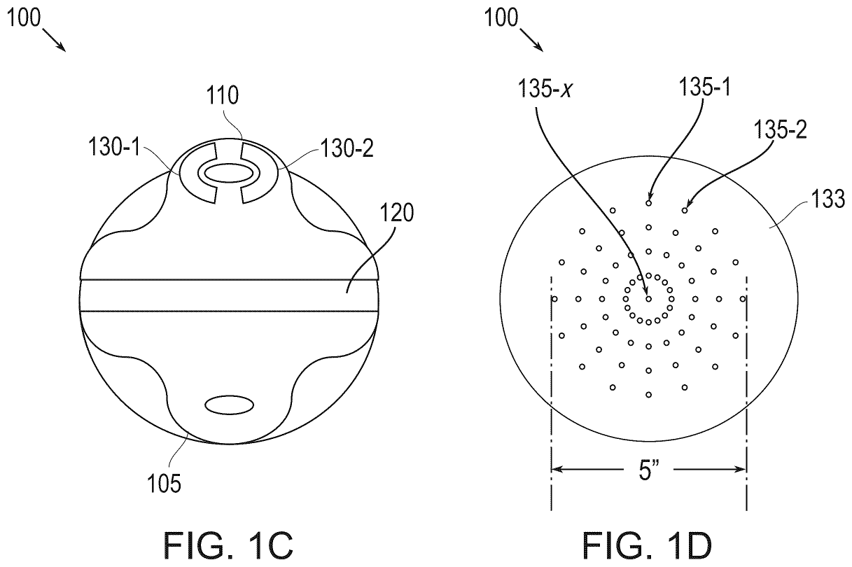

FIGS. 1A, 1B, 1C, and 1D are respective views illustrating a cooling substance container bag 100 according to one example implementation of the present disclosure. It is noted that these drawing figures show the structural elements of container 100 and are not shown to scale. Container 100 can have various shapes and/or sizes while incorporating the elements described in the following and illustrated in the drawing figures without departing from the spirit and scope of the present disclosure.

FIG. 1A is a front side view and FIG. 1B is a rear side view of container 100 according to an embodiment of the present disclosure. As illustrated in FIGS. 1A and 1B, container 100 can be in the shape of a bag with handles 105 and 110 on a top portion thereof. According to one example implementation of the present disclosure, container 100 is made of an insulating material, such as neoprene or the like. As illustrated in FIGS. 1A and 1B, container 100 includes a front side wall 113-1 and a rear side wall 113-2, which are about 12 inches (″) in height, about 8″ in width. In embodiments, front side wall 113-1 and rear side wall 113-2 can form a unitary cylindrical side wall 113 or can be formed by joining respective panels of material to form plural side walls 113-n (n>1). According to one example embodiment, sidewalls 113-1 and 113-2 are both made of an insulating material—for example, neoprene, or the like—to prevent heat transfer from the exterior into the interior of container 100, where a cooling substance can be contained. According to one example embodiment, the insulating material, for example neoprene, is about 8 millimeters (mm) in thickness. Thus, side walls 113-1 and 113-2 and a bottom wall 133 (shown in FIG. 1D) together form an interior compartment (not shown) of container 100 that is adapted to contain a cooling substance. In embodiments, container 100 can include additional interior layers (not shown) that can form additional internal walls (not shown) and to thereby form one or more additional interior compartments, pockets, or the like, in container 100. In embodiments, container 100 can have different height, width, and thickness dimensions without departing from the spirit and scope of the present disclosure.

Container 100 incorporates an opening in the top portion between handles 105 and 110, as illustrated in FIG. 1C and as described below. According to one example implementation, handles 105 and 110 are sized and disposed at the top portion of container 100 to provide for convenient handling—as an example, handles 105 and 110 each incorporate a through hole (111-1 and 111-2) that is about 2.5″ in width and about 1.25″ from the outer sides of the respective handle (105 and 110) for ease of carrying container 100. Thus, container 100 can be conveniently carried to an ice maker or cooling substance storage freezer (not shown), filled with a cooling substance (or ice), and returned to a water source for facilitating a cold therapy shower. Referring back to FIG. 1A, handle 105 includes fastener areas 115-1 and 115-2 on an outer side thereof. Fastener areas 115-1 and 115-2 incorporate hook and loop type fasteners—e.g., Velcro® or the like—to fasten to corresponding areas on handle 110 (e.g., 130-1 and 130-2 shown in FIG. 1C) so as to wrap handles 105 and 110 around a water source, such as a shower head, and to thereby attach, or hang, container 100 to the water source, as described below. According to one example implementation, fastener areas 115-1 and 115-2 each have an area of about 7.5 square inches (for a total area of about 15 square inches), which ensures sufficient attachment strength to secure container 100 to a water source. In embodiments, fastener areas 115 can have a total area of about 10-20 square inches. In certain embodiments, a singular connected fastener area 115 can be disposed around an aperture of handle 105.

FIG. 1C is a top view of container 100 according to an embodiment of the present disclosure. As illustrated in FIG. 1C, handles 105 and 110 can be pulled apart in respective front and rear directions to facilitate and/or widen an opening 120 in a top portion of container 100. In embodiments, container 100 can include a closing mechanism (not shown), such as a zipper or the like, for closing container 100 and thereby sealing opening 120. As illustrated in FIG. 1C, a cooling substance, such as ice or the like, can be placed into container 100 through opening 120. For example, ice cubes from a conventional freezer at home, ice from an ice maker, packaged ice, or one or more reusable freezer ice packs can be placed in container 100 through opening 120. In embodiments, one or more pockets or additional compartments (not shown) can be formed in the interior of container 100 for accommodating the cooling substance. For example, one or more pockets can be incorporated to the interior surface(s) of side wall(s) 113 or bottom wall 133 and sized to fit a respective reusable freezer ice pack on the interior of container 100. Additionally, the one or more interior pockets, or compartments, (not shown) can be sized and adapted to contain ice or ice cubes from an ice maker. In embodiments, the one or more pockets (not shown) can be made from the same, or a different, material as the remainder of container 100, which can be neoprene or the like as described above. In embodiments, the one or more pockets (not shown) can each incorporate one or more apertures to allow water from the water source to enter and/or exit to thereby allow the cooling substance to cool the water from the water source. In embodiments, the one or more pockets (not shown) can be made from a non-insulting material, such as a polyethylene layer or the like, to allow heat transfer from the water to the cooling substance.

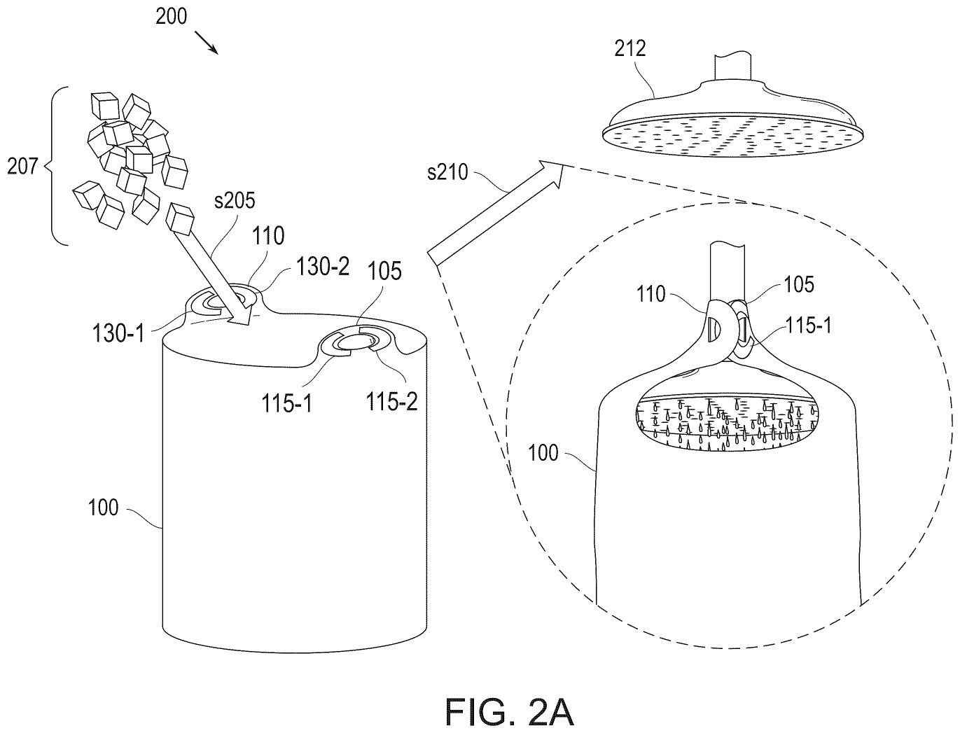

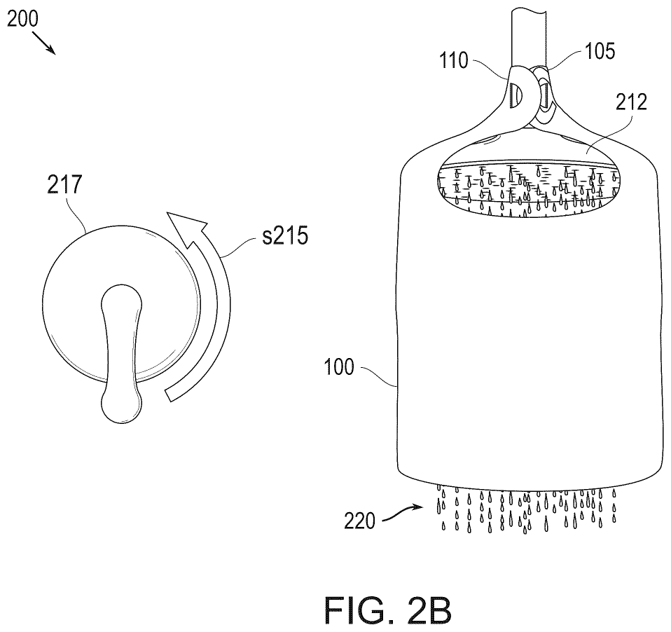

Referring back to FIG. 1C, handle 110 includes fastener areas 130-1 and 130-2 on an inner side thereof. Fastener areas 130-1 and 130-2 incorporate hook and loop type fasteners—e.g., Velcro® or the like—to fasten to corresponding areas on handle 105 (e.g., 115-1 and 115-2 shown in FIG. 1A) so as to wrap handles 105 and 110 around a water source, such as a shower head, and to thereby attach, or hang, container 100 to the water source. According to one example implementation, fastener areas 130-1 and 130-2 each have an area of about 7.5 square inches (for a total area of about 15 square inches), which ensures sufficient attachment strength to secure container 100 to a water source. In embodiments, fastener areas 130 can have a total area of about 10-20 square inches. In certain embodiments, a singular connected fastener area 130 can be disposed around an aperture of handle 105. One of ordinary skill in the art would appreciate that any fastening mechanism can be used in place of fastener areas 115 and 130 for attaching container 100 to a water source (e.g., 212 in FIG. 2A), including without limitation, buckles, straps, suction cups, and the like. In embodiments, a closing mechanism (not shown) can be closed partially around the water source when container 100 is attached to the water source so as to form at least a partial seal around the water source and to thereby ensure that a substantial portion of the water from the water source enters opening 120 to be cooled by the cooling substance in container 100.

FIG. 1D is a bottom view of container 100 according to an embodiment of the present disclosure. As illustrated in FIG. 1D, container 100 incorporates a circular bottom wall 133 according to one example embodiment of the present disclosure. According to one embodiment, as with side walls 113-1 and 113-2, bottom wall 133 is made of an insulating material, for example neoprene (and, for example, at 8 mm thickness), to prevent heat transfer from the exterior into the interior of container 100, where a cooling substance can be contained. In embodiments, side wall(s) 113 and bottom wall 133 can be formed by respective panels of material that are joined together or can be integrated with one another by a singular panel of material. Thus, as described above, side walls 113-1 and 113-2 and bottom wall 133 together form an interior compartment (not shown) of container 100 that is adapted to contain a cooling substance. Accordingly, the interior compartment (not shown) is substantially cylindrical in shape, although side walls 113 and bottom wall 133 are made of flexible material, such as neoprene. As also described before, in embodiments, container 100 can include additional interior layers (not shown) that can form additional internal walls (not shown) and to thereby form one or more additional interior compartments, pockets, or the like, in container 100.

As further illustrated in FIG. 1D, bottom wall 133 of container 100 includes a plurality of apertures 135, which are arranged in one or more patterns. According to one example implementation of the present disclosure, each aperture 135-1, 135-2, . . . , 135-x (x=an integer>2) is about ⅛ inches in diameter and x is 129, where the ⅛-inch apertures are arranged in approximately equidistant concentric circles around a central aperture 135-x that is disposed in the center of bottom wall 133. As illustrated in FIG. 1D, four concentric circles of apertures 135, which are equidistant and radially aligned with one another, are arranged on bottom wall 133 to form 128 apertures (135) in addition to the central aperture 135-x Thus, according to an example implementation, apertures 135 are arranged with radial symmetry to ensure even distribution for water exit flow. Additionally, as illustrated in FIG. 1D, the pattern of apertures 135 has an outer diameter of about 5 inches according to one example implementation of the present disclosure. In embodiments, apertures 135 can be formed with misalignments, other planar symmetries, etc. As described before, bottom wall 133 is about 8 inches in diameter according to one embodiment of the present disclosure. Thus, a ratio of the collective aperture area to the overall bottom wall area is approximately 1:32 according to one example implementation of the present disclosure. A ratio of the diameter of the aperture pattern and the diameter of the bottom wall 133 is about 5:8- or a ratio an area spanned by the aperture pattern and the overall bottom wall area is about 1:2.6. In embodiments, differently sized and arranged apertures, with different area and/or dimension ratios, can be disposed on bottom wall 133 without departing from the spirit and scope of the present disclosure. For example, just as there are different shower head arrangements, so too can bottom wall 133 incorporate different aperture sizes and patterns to emulate different types of shower heads, or other types of water sources, and their respective flow characteristics and rates.

FIGS. 2A and 2B together illustrate a process of utilizing container 100 to initiate a cold therapy shower according to one example implementation of the present disclosure. As illustrated in FIG. 2A, at an initial step s205, a cooling substance, such as ice cubes, 207 is placed into container 100—for example, through opening 120 illustrated in FIG. 1C. Once a sufficient amount of cooling substance 207 is placed into container 100, process 200 next proceeds to a step s210, where container 100 is attached to a water source 212, such as a shower head or the like. According to one example implementation, handles 105 and 110 are pulled up on opposing sides of water source 212 so that opening 120 (shown in FIG. 1C) is aligned with the outlet of water source 212. Once opening 120 is aligned, handle 110 is folded over handle 105 around water source 212 so that fastener areas 130-1 and 130-2 on the inner side of handle 110 can be fastened to the fastener areas 115-1 and 115-2 on the outer side of handle 105. In this manner, container 100 is attached to water source 212 with opening 120 aligned to the outlet of water source 212.

As illustrated in FIG. 2B, at a next step s215, water source 212 is switched on for example, by rotating a dial or knob 217. The water from water source 212 enters container 100 through opening 120 (FIG. 1C) and is cooled by cooling substance 207 before exiting container 100 through the bottom apertures 135 (FIG. 1D) as a cooled water stream 220. In embodiments, side walls 113 and/or bottom wall 133 can include one or more rigid elements (not shown), which can be made from a polymer, a metal, or the like, for maintaining a shape and corresponding water flow characteristics—e.g., flow rate and direction—for cooled water stream 220. Correspondingly, in embodiments, container 100 can incorporate internal channels, pockets, walls, holes, and the like, (not shown) to direct water flow from water source 212 for cooling and/or to maintain exit flow characteristics for cooled water stream 220.

Thus, advantageously, container 100 according to the present disclosure provides for conveniently facilitating cold therapy showers without the need for dedicated space or hardware. Additionally, container 100 provides for facilitating cold therapy showers while traveling wherever a shower head, or a suitable water source, is available.

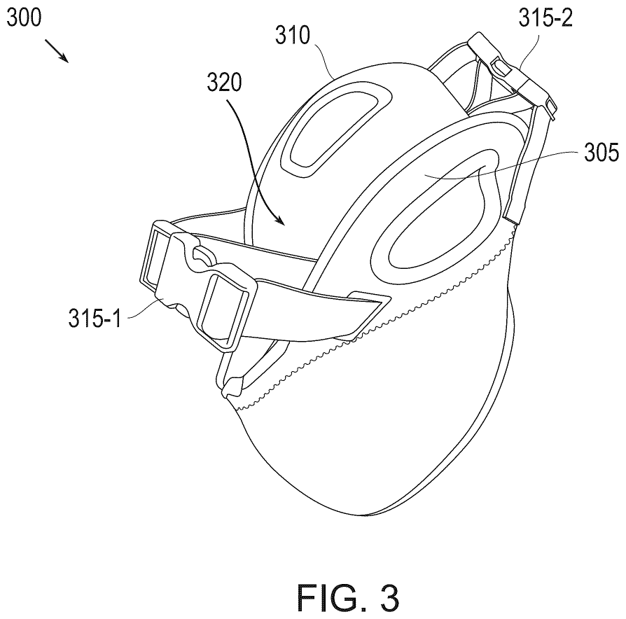

FIG. 3 illustrates a container 300 according to another embodiment of the present disclosure. As shown in FIG. 3, container 300 generally conforms to container 100 but incorporates side squeeze release buckle fasteners 315-1 and 315-2 in place of fastener areas 115-1, 115-2, 130-1, and 130-2. Thus, handles 305 and 310 can be pulled apart around a water source (212) to align opening 320 with the water source (212) and fasteners 315-1 and 315-2 can be fastened to one another around the water source (212) to secure container 300 in place to facilitate a cold therapy shower. In embodiments, alternative fastening mechanisms, such as loop back hook and loop fasteners or the like, can also be used to secure container 100 or 300 to a water source (212).

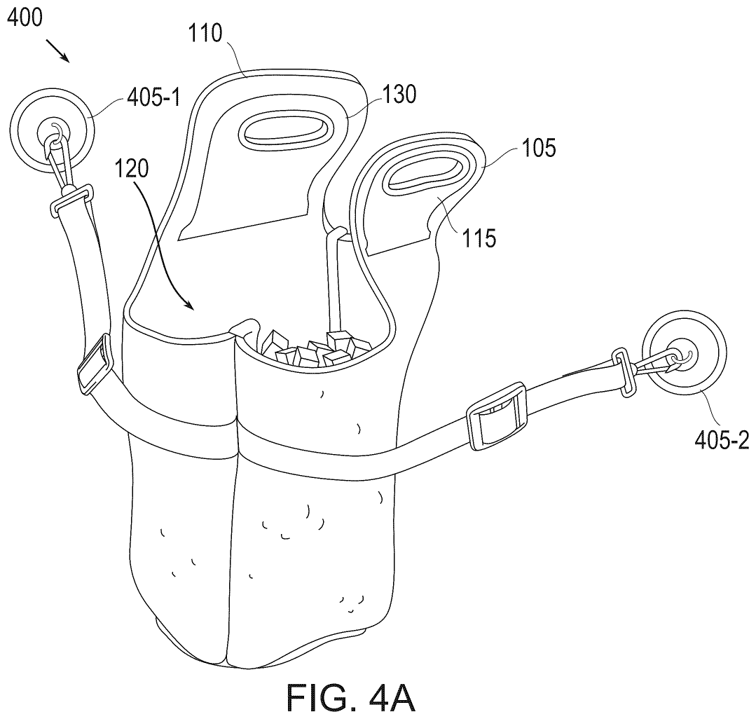

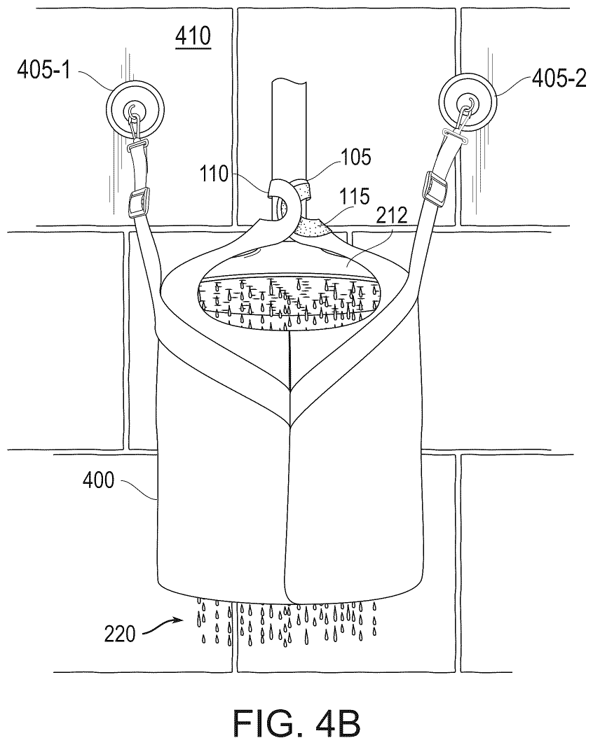

FIG. 4A illustrates a container 400 according to another embodiment of the present disclosure. As illustrated in FIG. 4A, container 400 generally conforms to container 100 and container 300. The same reference numerals are used to denote corresponding elements. As an example, container 400 incorporates fastener areas 115 and 130 on handles 105 and 110, respectively. As shown in FIG. 4A, container 400 also incorporates additional fasteners 405-1 and 405-2. In one or more example implementations, fasteners 405-1 and 405-2 are suction cups attached (e.g., via adjustable straps) to container 400 for fastening to a wall proximate to a water source (e.g., shower head 212). In certain embodiments, one or more fasteners 405, including without limitation, buckles, straps, hook and loop fasteners, and the like, can be incorporated to container 400 for fastening to any structure proximate to a water source (e.g., 212), such as, an enclosure, a curtain rod, a door, or the like.

FIG. 4B illustrates container 400 in use, where fasteners 405-1 and 405-2 are fastened to a shower wall 410 proximate to a water source (e.g., shower head) 212 to support the weight of container 400 while it is aligned with the water source 212, receives water via opening 120 for cooling, and dispenses cooled water 220 via apertures 135 (see FIG. 1D).

### EXAMPLES

It is understood that an average ice cube can range from 0.75 to 1.5 cubic inches (and can be about ½ ounce in weight each) and, thus, according to one example implementation, container 100 can be filled with about 5 to 12 pounds (lbs.) or about 9 to 11 lbs.—of ice at step s205 described above. The temperature of cold water from a conventional shower has been measured at about 709 Fahrenheit (F) in other words, an entry temperature of water from a water source into container 100 has been measured to be about 709 F. With container 100 attached to a shower head (212) in the manner described above, a cooled water stream 220 has been measured to be at about 509 F—in other words, an exit temperature of cooled water stream 220 from container 100 has been measured at about 509 F. A filled container 100 has been measured to provide cooled water stream 220 at about the measured 50° F. for a cold therapy shower lasting about 3-5 minutes, or about 4 minutes.

The headings used herein are for organizational purposes only and are not meant to be used to limit the scope of the description or the claims. As used throughout this application, the words “may” and “can” are used in a permissive sense (i.e., meaning having the potential to), rather than the mandatory sense (i.e., meaning must). To facilitate understanding, like reference numerals have been used, where possible, to designate like elements common to the figures. In certain instances, a numeral suffix following a dash ( . . . −1) denotes a specific example of an element marked by a particular reference numeral (e.g., 210-1). Description of elements with references to the base reference numerals (e.g., 210) also refer to all specific examples with such numeral suffixes (e.g., 210-1), and vice versa.

### Exemplary Products, Systems and Methods are Set Out in the Following Items

1. A container, comprising:
an opening in a top portion sized and adapted to receive water from a water source;
   one or more side walls and a bottom wall below the opening, said one or more side walls and the bottom wall forming at least one interior compartment; a plurality of apertures on the bottom wall; and
   one or more fasteners in the top portion above the opening, the one or more fasteners being adapted to attach the container to the water source,
   wherein the one or more side walls incorporate an insulating material and the at least one interior compartment is thereby adapted to contain a cooling substance for cooling the water received from the water source and to allow cooled water to exit the at least one interior compartment through the plurality of apertures on the bottom wall.

2. The container of item 1, further comprising a plurality of handles, wherein the one or more fasteners comprise a plurality of hook and loop fastener areas incorporated on respective ones of the plurality of handles of the container.

3. The container of any one of the preceding items, wherein a first hook and loop fastener area is disposed on an inner side of a first one of the plurality handles and a second hook and loop fastener area is disposed on an outer side of a second one of the plurality of handles.

4. The container of any one of the preceding items, wherein the plurality apertures are arranged in a radially symmetrical pattern.

5. The container of any one of the preceding items, wherein the plurality of apertures are arranged in a radially aligned pattern.

6. The container of any one of the preceding items, wherein the bottom wall is circular in shape.

7. The container of any one of the preceding items, further comprising one or more additional fasteners adapted to fasten to a structure proximate to the water source.

8. The container of any one of the preceding items, wherein the one or more additional fasteners comprise one or more suction cups adapted to fasten to a wall proximate to the water source.

9. A method of initiating a cold therapy shower using a container according to item 1, said method comprising:
placing a cooling substance into the at least one interior compartment through the opening;
attaching the container to the water source using the one or more fasteners while aligning the opening to water source; and
initiating a water flow from the water source into the opening.

10. The method of item 9, wherein the cooling substance comprises about 5 to 12 pounds of ice.

11. The method of any one of items 9 and 10, wherein an entry temperature of the water flow from the water source is approximately 70 degrees Fahrenheit.

12. The method of any one of items 9, 10 and 11, wherein an exit temperature of cooled water flowing from at least one of the plurality of apertures of the container is approximately 50 degrees Fahrenheit.

13. The method of any one of items 9-12, wherein the exit temperature is maintained for about 3 to 5 minutes.

14. The method of any one of items 9-13, wherein the exit temperature is maintained for about 4 minutes.

It is to be further understood that like or similar numerals in the drawings represent like or similar elements through the several figures, and that not all components or steps described and illustrated with reference to the figures are required for all embodiments or arrangements.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof, and are meant to encompass the items listed thereafter and equivalents thereof as well as additional items.

Terms of orientation are used herein merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third) is for distinction and not counting. For example, the use of “third” does not imply there is a corresponding “first” or “second.” Also, the phraseology and terminology used herein is for the purpose of description and should not be regarded as limiting.

While the disclosure has described several example implementations, it will be understood by those skilled in the art that various changes can be made, and equivalents can be substituted for elements thereof, without departing from the spirit and scope of the disclosure. In addition, many modifications will be appreciated by those skilled in the art to adapt a particular instrument, situation, or material to embodiments of the disclosure without departing from the essential scope thereof. Therefore, it is intended that the disclosure not be limited to the particular embodiments disclosed, or to the best mode contemplated for carrying out this disclosure, but that the disclosure will include all embodiments falling within the scope of the appended claims.

The subject matter described above is provided by way of illustration only and should not be construed as limiting. Various modifications and changes can be made to the subject matter described herein without following the example embodiments and applications illustrated and described, and without departing from the true spirit and scope encompassed by the present disclosure, which is defined by the set of recitations in the following claims and by structures and functions or steps which are equivalent to these recitations.

1. A container, comprising:
an opening in a top portion sized and adapted to receive one or more units of a cooling substance and to receive water from a water source;
one or more side walls and a bottom wall below the opening, said one or more side walls and the bottom wall forming at least one interior compartment;
a plurality of apertures on the bottom wall; and
one or more fasteners in the top portion above the opening, the one or more fasteners being adapted to attach the container to the water source,
wherein the one or more side walls incorporate an insulating material and the at least one interior compartment is thereby adapted to contain the one or more units of the cooling substance received via the opening for cooling the water received from the water source and to allow cooled water to exit the at least one interior compartment through the plurality of apertures on the bottom wall.

2. The container of claim 1, further comprising a plurality of handles, wherein the one or more fasteners comprise a plurality of hook and loop fastener areas incorporated on respective ones of the plurality of handles of the container.

3. The container of claim 2, wherein a first hook and loop fastener area is disposed on an inner side of a first one of the plurality handles and a second hook and loop fastener area is disposed on an outer side of a second one of the plurality of handles.

4. The container of claim 1, wherein the plurality apertures are arranged in a radially symmetrical pattern.

5. The container of claim 4, wherein the plurality of apertures are arranged in a radially aligned pattern.

6. The container of claim 1, wherein the bottom wall is circular in shape.

7. The container of claim 1, further comprising one or more additional fasteners adapted to fasten to a structure proximate to the water source.

8. The container of claim 7, wherein the one or more additional fasteners comprise one or more suction cups adapted to fasten to a wall proximate to the water source.

9. A method of initiating a cold therapy shower using a container, wherein said container comprises:
an opening in a top portion sized and adapted to receive one or more units of a cooling substance and to receive water from a water source;
one or more side walls and a bottom wall below the opening, said one or more side walls and the bottom wall forming at least one interior compartment;
a plurality of apertures on the bottom wall; and
one or more fasteners in the top portion above the opening, the one or more fasteners being adapted to attach the container to the water source,
wherein the one or more side walls incorporate an insulating material and the at least one interior compartment is thereby adapted to contain the one or more units of the cooling substance received via the opening for cooling the water received from the water source and to allow cooled water to exit the at least one interior compartment through the plurality of apertures on the bottom wall;
said method comprising:
placing the one or more units of the cooling substance into the at least one interior compartment through the opening;
attaching the container to the water source using the one or more fasteners while aligning the opening to the water source; and
initiating a water flow from the water source into the opening.

10. The method of claim 9, wherein the one or more units of the cooling substance comprises about 5 to 12 pounds of ice.

11. The method of claim 9, wherein an entry temperature of the water flow from the water source is approximately 70 degrees Fahrenheit.

12. The method of claim 9, wherein an exit temperature of cooled water flowing from at least one of the plurality of apertures of the container is approximately 50 degrees Fahrenheit.

13. The method of claim 12, wherein the exit temperature is maintained for about 3 to 5 minutes.

14. The method of claim 13, wherein the exit temperature is maintained for about 4 minutes.
